(19) United States
(12) Patent Application Publication
(10) Pub. No.: US 2020/0233975 A1
(43) Pub. Date: Jul. 23, 2020

(54) SECURE MANAGEMENT OF USER INFORMATION USING TOKENIZATION AND TOKEN STATES

(71) Applicant: EMC IP Holding Company LLC, Hopkinton, MA (US)

(72) Inventors: Joshua Rosenthol, Centreville, VA (US); Robert W. Griffin, Hollis, NH (US)

(21) Appl. No.: 16/839,491

(22) Filed: Apr. 3, 2020

Related U.S. Application Data
(63) Continuation of application No. 12/730,529, filed on Mar. 24, 2010.

(51) Int. Cl. G06F 21/62; G06Q 50/26; G06Q 30/02; G06Q 20/34; G16H 10/60; G06F 16/25; G06F 7/58; G06F 16/23; G06F 21/60; H04L 9/08; H04L 9/32

(52) U.S. Cl. CPC G06F 21/6245; G06Q 50/265; G06Q 30/0201; G06Q 20/34; G16H 10/60; H04L 9/3213; G06F 7/58; G06F 16/2379; G06F 21/602; H04L 9/0869; G06F 16/25

(57) ABSTRACT

A technique smartly manages user information. The technique involves receiving, using an input device, an input value from the user during a user transaction. The technique further involves applying, using electronic circuitry, an algorithm to generate a token on behalf of the user. The algorithm is arranged to provide tokens in a non-reproducible manner to prevent discovery of input values based on the tokens. The technique further involves associating the token value with the input value and collecting user information and associating the user information with the token during a time range which includes periods of transaction inactivity and further transaction activity by the user. Along these lines, the technique is capable of implementing mutually exclusive token states (e.g., “active”, “inactive”, “deactivated” and “compromised”) to smartly coordinate and maintain certain user information.

### CROSS REFERENCE TO RELATED APPLICATIONS

The present application is a Continuation of U.S. patent application Ser. No. 12/730,529, filed Mar. 24, 2010, all disclosures of which are hereby included by reference herein.

### BACKGROUND

When a customer completes a purchase with a retailer using a credit card, the retailer typically reads the customer's credit card information and provides that credit card information to a credit card transaction clearing center in order to receive payment from that customer's credit card account. Such a purchase may be made in person at a retail store. Alternatively, such a purchase may be made remotely (e.g., via an online purchase on the Internet).

In some situations, the retailer may save the customer's credit card information even after payment is received through the transaction clearing center. For example, it may be appropriate in some situations for the customer to return a purchased item and receive a refund. In such a situation, the retailer may be able provide the refund back to the customer's credit card account using the saved credit card information of the customer.

Regulations and guidelines exist for the protection of non-public information (NPI), personally identifiable information (PH), and payment card industry (PCI) information. Such regulations and guidelines define, among other things, how and how long a customer's credit card information can be stored by third parties such as a retailer. Similarly, there are regulations and guidelines regarding the handling of protected health information (PHI).

### SUMMARY

It should be understood that the task of handling sensitive non-public information among different entities may be complex. For example, when a retailer obtains credit card information from customers during credit card purchases, the retailer may wish to gather and save additional customer information. Along these lines, the retailer may wish to accumulate, for each of its credit card customers, purchase habit data such as a list of routinely purchased items, the average purchase amount, the frequency of purchases, whether that credit card customer uses coupons, and so on. Similarly, when a hospital admits a patient, the hospital may wish to track information about that patient for future reference.

However, data security regulations may restrict how and how long certain security sensitive information is stored. Using the retail example, the transaction processing department of the retailer may have gathered purchase habit data on its credit card customers and may be able to share this purchase habit data with the advertising and promotional department. Yet, in order to comply with certain data security rules, it would be careless for the transaction processing department to simply share all of its customer information with the advertising and promotional department. Rather, the prudent course of action is for the transaction processing department to share only general customer information (e.g., perhaps customer names, phone numbers, zip codes, etc.) as well as the gathered purchase habit data, but withhold the customer credit card information.

Furthermore, to remain compliant with data security regulations, the retailer may be required to destroy certain customer information as time passes. Such customer information may include the customer credit card information of very old transactions and the general customer information (e.g., perhaps customer names, phone numbers, zip codes, etc.) gathered during these very old transactions.

Unfortunately, it is often difficult for departments within a company to comprehensively track and destroy customer information as needed. For example, the transaction processing department of the retailer may successfully destroy its records of old customer information as time passes. However, if the advertising and promotional department still maintains the earlier-shared general customer information, the retailer may be out of compliance with particular data security regulations.

In some instances, companies may attempt to employ a process commonly referred to as “tokenization” to improve data security. Tokenization involves replacing sensitive data with unique identification symbols (i.e., tokens) that may be meaningless to an outsider (e.g., a security hacker). For example, a retailer may be able to save tokens in place of actual customer credit card information from its credit card sales. Unfortunately, there is still no convenient way to efficiently and effectively collect and manage customer information over an extended period of time in situations involving the use of tokens. Along these lines, a standard methodology for managing only old and new customer credit card information while remaining compliant with data security regulations is still unavailable.

In contrast, improved techniques are directed to managing user information (e.g., customer purchase habits) in an electronic system which smartly applies a tokenization process over a time range which includes periods of user transaction activity and inactivity. In particular, the system is equipped to associate initial user information (e.g., initial purchase information) with a token, and subsequently associate additional user information (e.g., subsequent purchase information) with the same token even if an extended amount of time has passed. Such operation is coordinated by rotating the token through various states of a token lifecycle. In some arrangements, the system implements formal mutually exclusive token states (e.g., “active”, “inactive”, “deactivated” and “compromised”) to smartly manage the user information.

Some embodiments are directed to a method of managing user information. The method includes receiving, using an input device (e.g., a credit card reader), an input value (e.g., credit card information) from the user during a user transaction (e.g., a credit card purchase). The method further includes applying, using electronic circuitry, a random number generation algorithm to the input value to generate a token on behalf of the user. The result of the random number generation algorithm (e.g., a cryptographic function) is then associated with the input value and this information is stored securely, preventing discovery of the input values based on the tokens. There is no cryptographic relationship between the input value and the token, thus removing all cryptographic-based vectors of attack for discovering the input value from the token. The method further includes collecting user information (e.g., purchase habit data) and associating the user information with the token during a time range which includes periods of transaction inactivity and further transaction activity by the user.

Additionally, some embodiments are directed to a computer program product including a computer readable storage medium storing instructions which cause a computer to perform the method of managing user information. In some arrangements, the instructions are compiled and linked executable code. In other arrangements, the instructions are scripts or rules which are dynamically translated and then performed by the computer.

Furthermore, some embodiments are directed to an electronic system which performs the method of managing user information. Here, various components of the electronic system communicate and exchange electronic signals to carry out the method of managing user information.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a block diagram of an electronic system which manages user information by smartly utilizing tokenization.

FIG. 2 is a block diagram illustrating certain interactions between various components of the electronic system of FIG. 1.

FIG. 3 is a state diagram illustrating various mutually exclusive states for tokens utilized by the electronic system of FIG. 1.

FIG. 4 is a flowchart of a procedure which is performed by the electronic system of FIG. 1.

FIG. 5 is a block diagram of a suitable structure for a token database of the electronic system of FIG. 1.

### DETAILED DESCRIPTION

An improved technique is directed to managing user information in an electronic system which smartly applies a tokenization process over an extended time range including episodes of user transaction activity and inactivity. During operation, the system associates initial user information (e.g., data regarding a transaction) with a token, and subsequently associates additional user information (e.g., data regarding further transactions) with the token even if a substantial amount of time has passed since the last transaction. Such operation is coordinated by rotating the token through different states of a token lifecycle. For example, the system is capable of implementing formal mutually exclusive token states such as “active”, “inactive”, “deactivated” and “compromised” to smartly manage the user information during the extended time range.

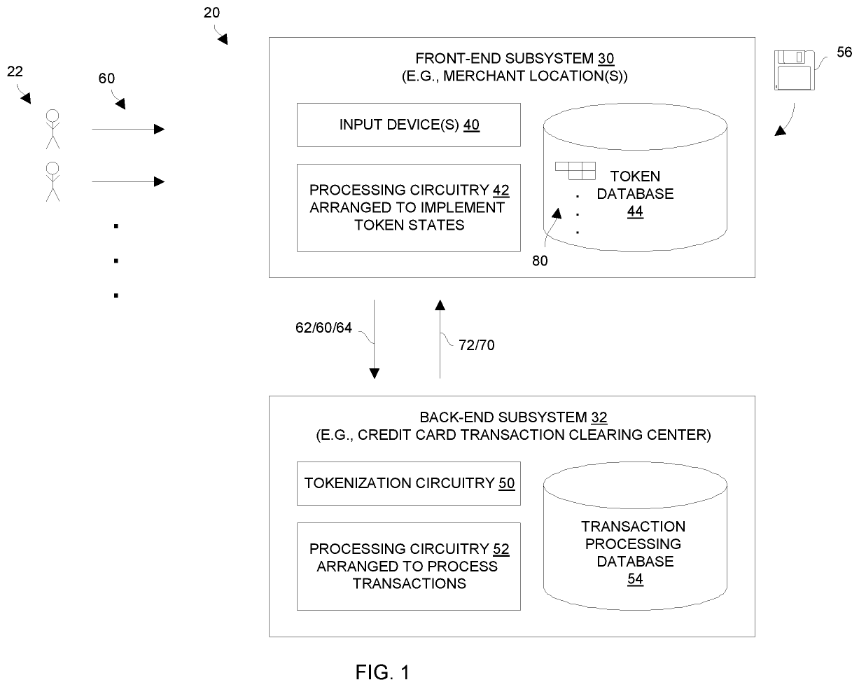

FIG. 1 is a block diagram of an electronic system 20 which effectively and efficiently processes and manages data relating to user transactions (e.g., credit card purchases) using tokens and token states. As will be explained in further detail below, each token uniquely corresponds to a sequence of symbols which uniquely identify a particular user 22 (e.g., a credit card number).

As shown in FIG. 1, the electronic system 20 includes a front-end subsystem 30 and a back-end subsystem 32. The front-end subsystem 30 and the back-end subsystem 32 may reside in different locations, and communicate over a computer network or similar communications medium. The front-end subsystem 30 includes a set of input devices 40, processing circuitry 42 arranged to implement token states, and a token database 44. The back-end subsystem 32 includes tokenization circuitry 50, processing circuitry 52 arranged to process transactions, and a transaction processing database 54.

It should be understood that one or more of the various components of the system 20 may be implemented on a computerized device. That is, such a component (e.g., the processing circuitry 42 of the front-end subsystem 30) includes a microprocessor and memory which stores an application. As the microprocessor executes the application from the memory, the microprocessor carries out specialized operational tasks to transform data. In these situations, the application is capable of being delivered to the component via a computer program product 56 which includes a computer readable storage medium storing instructions (e.g., executable code) which control the operation of the microprocessor. Examples of suitable computer readable storage media for the computer program product 56 include CD-ROM, flash memory, magnetic diskettes, magnetic tape, and the like.

During system operation, the front-end subsystem 30 is equipped to associate (or link) user transaction information with tokens rather than actual user identification data which may be more security-sensitive and which could otherwise pose a security issue (e.g., credit card information). Since the tokens are provided with states as mentioned above, the front-end subsystem 30 is able to competently store and manage the token and the user transaction information over an extended period of time (e.g., many years) while maintaining compliance with rules and regulations regarding data security.

Along these lines, the front-end subsystem 30 is constructed and arranged to receive input values 60 from the users 22 as the users 22 engage in the user transactions. The input values 60 uniquely identify the users 22 and may be security-sensitive (e.g., vulnerable to attackers). In particular, an input device 40 of the front-end subsystem 30 takes an input value 60 uniquely identifying a particular user 22 and sends a transaction message 62 to the back-end subsystem 32 for processing. The transaction message 62 includes the input value 60 and a transaction description 64 identifying particular details of the transaction (e.g., an amount of the transaction, a date stamp identifying the date and time of day, etc.).

The tokenization circuitry 50 of the back-end subsystem 32 receives the transaction message 62, and applies a random number generation algorithm to generate a user token 70. This token 70 is then associated with the user input value 60 in the transaction message 62, and the tokenization circuitry 50 returns a response message 72 containing the user token 70 to the front-end subsystem 30. Additionally, the processing circuitry 52 of the back-end subsystem 32 performs transactional operations based on the transaction description 64 to process the transaction, and stores the processing results in the transaction processing database 54.

Upon receipt of the user token 70 from the back-end subsystem 32, the processing circuitry 42 of the front-end system 30 checks the token database 44 to determine whether the user token 70 already exists in the token database 44 and is in a useable state, e.g., “active”, “inactive”, or “deactivated”. If the token 70 does not already exist, the processing circuitry 42 adds a new entry 80 into the token 70 to the token database 44. Next, the processing circuitry 42 links the various information regarding the transaction to the user token 70 (e.g., the processing circuitry 42 can store non-sensitive information with the user token 70 in the token database 44 as well), and sets the state of the token 70 to the active state.

However, if an entry 80 for the user token 70 already exists in the token database 44 and the entry 80 indicates that the token 70 is in a useable state, the processing circuitry 42 simply links the various information regarding the transaction with the user token 70 (e.g., non-sensitive transaction details, date and time information, a location of the particular input device 40, and operator details, etc.) and sets the state of the token to the active state if the state was not already active. Such a situation exists when the user 22 has performed a transaction with the front-end subsystem 30 at least once before using the same input value 60.

Furthermore, if an entry 80 for the user token 70 already exists in the token database 44 but indicates that the token 70 is in a non-usable state (e.g., “compromised”), the processing circuitry 42 performs a specialized task depending on the particular application. For example, the processing circuitry 42 may output a warning to warn the user 22 or the operator of the front-end subsystem 30. Alternatively, the processing circuitry 42 may further communicate with the back-end subsystem 32 to obtain a different user token 70 in a deterministic manner (e.g., by applying a second algorithm) and then perform the information linking process using the different user token 70.

It should be understood that the processing circuitry 42 routinely analyzes the entries 80 of the token database 44 to update the states of the user tokens 70 stored therein. That is, the processing circuitry 42 periodically readjusts (e.g., daily, monthly, etc.) the token states of the user tokens 70 within the token database 44 to accurately reflect the passage of time. Along these lines, it should be understood that, as time passes, certain time period thresholds may pass in which there is no additional transaction activity for a particular user token 70 stored in the token database 44. As these time period thresholds are met, the processing circuitry 42 changes the token state of the token 70 to reflect the current situation.

For example, suppose that a particular token 70 is set to the active state due to some transaction activity associated with that token 70. If a first time threshold (e.g., six months from the last transaction) passes with no further transaction activity linked to that token 70, the processing circuitry 42 changes the state from active to inactive. Similarly, if a second time threshold (e.g., two years months from the last transaction) passes with no further transaction activity linked to that token 70, the processing circuitry 42 changes the state from inactive to deactivated.

However, once the existing token 70 is on one of these non-active states, the processing circuitry 42 changes the state back to active upon the next transaction activity linked to that token 70. Such operation is beneficial over losing access to the user transaction history which frequently occurs in conventional transaction systems (e.g., due to data deletion in order to comply with data retention regulations). As a result, the electronic system 70 is well-suited for collecting user information and associating the user information with a token 70 during an extended time range which includes periods of transaction inactivity and further transaction activity by the user 22.

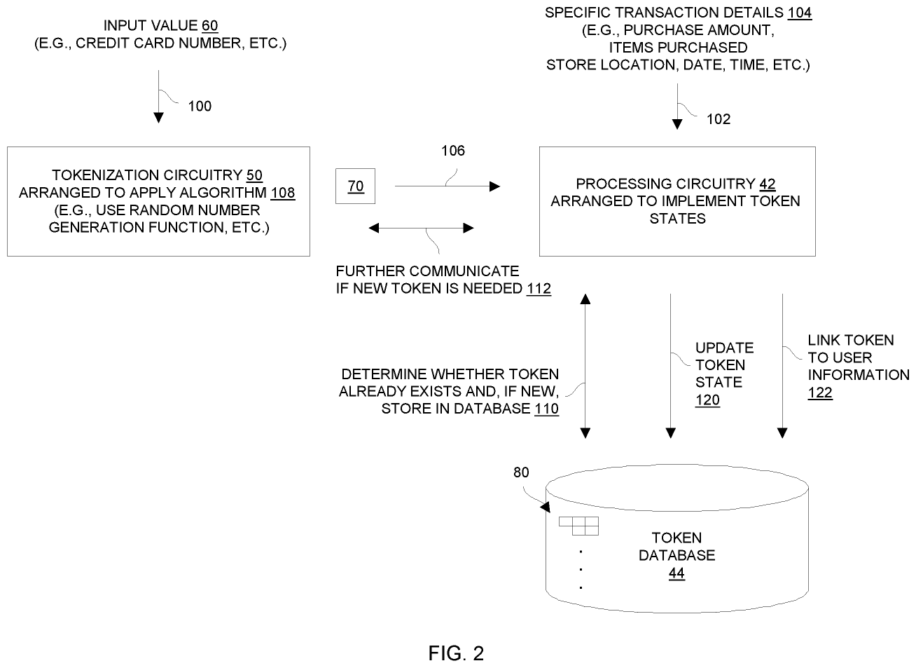

At this point, it should be understood that the above-described electronic system 20 is well-suited for a variety of transactional environments. For example, in a retail context, the front-end subsystem 30 may be a merchant's transaction processing system which generates credit card transactions as users 22 use their credit cards to make purchases, and the back-end subsystem 32 may be a credit card transaction clearing center which processes credit card transactions between the merchant and credit card accounts of the users 22. Along these lines, each input device 40 may be a cash register or credit card device at a store location of the merchant. Accordingly, the input devices 40 are constructed and arranged to obtain, as input values 60, credit card information when the users 22 (i.e., credit card customers) perform transactions with the merchant (e.g., make credit card purchases). In this situation, the tokens 70 returned by the back-end subsystem 32 uniquely identify the users 22 thus enabling the merchant to associate data regarding the credit card purchases (e.g., purchase habit data) with the tokens 70 rather than with the actual credit card information of the users 22. Further details will now be provided with reference to FIG. 2.

FIG. 2 is a block diagram illustrating certain interactions between particular components of the electronic system 20. For example, as shown by the arrow 100, the tokenization circuitry 50 of the back-end subsystem 32 receives the input value 60 gathered by an input device 40 of the front-end subsystem 30 from a user 22 at the time of a transaction (also see FIG. 1). Additionally, as shown by the arrow 102, the processing circuitry 42 of the front-end subsystem 30 receives specific transaction details 104 relating to the transaction.

Furthermore, as shown by the arrow 106, the tokenization circuitry 50 applies an algorithm 108 to generate a user token 70 in response to receipt of the input value 60, and provides the user token 70 to the processing circuitry 42. It should be understood that the algorithm 108 is a random number generation function, i.e., the input value 60 is not used to generate the user token 70. In some arrangements, the tokenization circuitry 50 is equipped to apply multiple random number generation algorithms 108 in the event that the initial application of an algorithm 108 poses certain difficulties.

As further shown in FIG. 2, the processing circuitry 42 of the front-end subsystem 30 receives the user token 70 (from the tokenization circuitry 50), and determines whether an entry 80 for the user token 70 already exists in the token database 44 as shown by the double-ended arrow 110. If the user token 110 has a “compromised” state, the processing circuitry 42 is capable of communicating with the tokenization circuitry 50 (e.g., by applying a second algorithm 108 to the input value 60) to obtain a new user token 70 which uniquely identifies the user 22 as shown by the double-ended arrow 112.

If an entry 80 for the user token 70 already exists in the token database 44 and has a non-compromised state (e.g., active, inactive, deactivated), the processing circuitry 42 updates the token state of that user token 70 to active as shown by the arrow 120, and links the specific transaction details 104 to the user token 70 as shown by the arrow 122.

If an entry 80 for the user token 70 is not in the token database 44, the processing circuitry 42 creates and stores a new entry 80 for the user token 70 in the token database 44 (arrow 110), sets the token state to active (arrow 120), and links the specific transaction details 104 to the user token 70 (arrow 122).

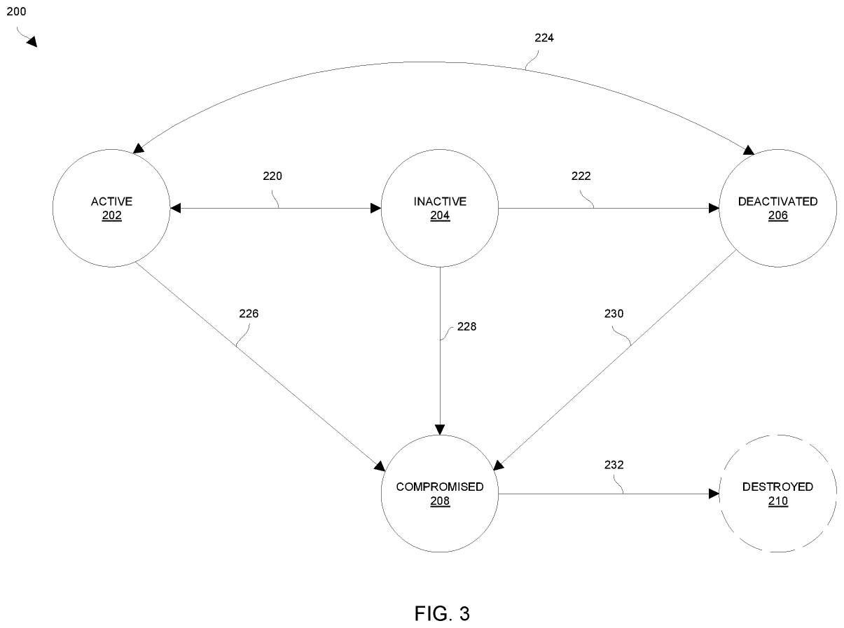

Furthermore, as mentioned earlier, the processing circuitry 42 periodically analyzes and, if necessary, adjusts the token states of the user tokens 70 in the token database 44 (see arrow 120 of FIG. 2). Such operation rotates the user tokens 70 through a lifecycle over time. Further details will now be provided with reference to FIG. 3.

FIG. 3 shows an example state diagram 200 for various mutually exclusive states of the user tokens 70 utilized by the electronic system 20. The state diagram 200 includes an active state 200, an inactive state 204, a deactivated state 206, a compromised state 208, and a destroyed state 210. In some arrangements, each token 70 is stored in a database entry 80 within the token database 44 (also see FIGS. 1 and 2), and a modifiable field of that database entry 80 stores a value indicative of the current token state.

When a user token 70 has the active state 202 (i.e., when the field of the database entry 80 for that user token 70 stores a value indicative of the active state 202), the user 22 corresponding to the user token 70 has performed transaction activity within a predefined amount of time, e.g., an active threshold time of six months. That is, the input value 60 from the user 22 has been processed within the active threshold time, and the user token 70 is considered to be an active token by the front-end circuitry 30. Such a determination is easily made by the processing circuitry 42 by scanning the transaction details 104 (FIG. 2) which are linked to that user token 70.

When a user token 70 has the inactive state 204, the user 22 corresponding to the user token 70 has not performed any transaction activity within the predefined amount of time but has performed transaction activity within another predefined amount of time, e.g., an inactive threshold time of two years. That is, the input value 60 from the user 22 has not been processed within the active threshold time but has been processed within the inactive threshold time, and the user token 70 is considered to be an inactive token by the front-end circuitry 30. As indicated by the double-ended arrow 220, a user token 70 is able transition between the active state 202 and the inactive state 204 in accordance with transaction activity and inactivity over time.

When a user token 70 has the deactivated state 206, the user 22 corresponding to the user token 70 has not performed any transaction activity within the inactive threshold time. As a result, the input value 60 from the user 22 has not been processed within the inactive threshold time, and the user token 70 is considered to be a deactivated token by the front-end circuitry 30. As indicated by the arrow 222, a user token 70 is able transition from the inactive state 204 to the deactivated state 206 when the time of transaction inactivity is greater than the inactive threshold time.

Another way for the user token 70 to have the deactivated state 206 is for an administrator to manually transition the user token 70 to the deactivated state (e.g., due to a purchase dispute). The arrow 224 points from the active state 202 to the deactivated state 206 to show that it is possible for the user token 70 to be purposefully transitioned from the active state 202 to the deactivated state 206 in this manner.

Furthermore, as further indicated by the arrow 224, a user token 70 is able transition from the deactivated state 206 back to the active state 202 in response to new transaction activity. As a result, relatively old user activity information may have been linked to the user token 70 and new activity information due to a new transaction following an extended period of inactivity can now be linked as well (e.g., old purchase habit data updated with new purchase habit data). Such a situation is unavailable in a conventional system in which the old user activity information linked to user sensitive information is lost due to deletion of the user sensitive information for compliance with data security rules and regulations.

As further shown in FIG. 3, when a user token 70 has the compromised state 208, the input value 60 used to generate the user token 70 has been correlated to an attacker. In such a situation, the input value 60 and the token 70 preferably are removed from the system 20, and the user 22 provides a new input value 60 for use within the system 20. Alternatively, the input value 60 is associated with a new token 70 (e.g., via application of a different algorithm), and the new token 70 is utilized within the system 20. It should be understood that, as shown by the arrows 226, 228, 230, a token 70 can enter the compromised state 208 from any of the earlier-mentioned states (e.g., due to flagging of the token 70 by an administrator).

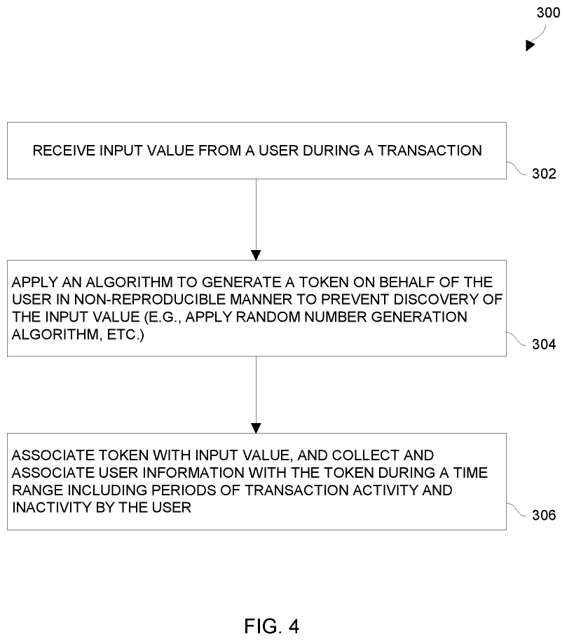

Once a user token 70 has the deactivated state 208, the processing circuitry 42 is able to transition the user token 70 to the destroyed state 210, as shown by the arrow 232, and permanently remove the user token 70 from the system 20 if desired. The destroyed state 210 is shown in dashed form (phantom) to indicate that it is a transitory state. In particular, once the user token 70 and its linked transaction data is removed from the system 20, there is no remaining reference to the user token 70. Further details will now be provided with reference to FIG. 4.

FIG. 4 is a flowchart showing a procedure 300 which is performed by the system 20. In step 302, an input device 40 of the system 20 receives an input value 60 from a user 22 during a user transaction. Recall that the front-end subsystem 30 may prepare and send a transaction message 62 which includes the input value 60 to the back-end subsystem 32 (also see FIG. 1).

In step 304, the tokenization circuitry 50 applies a random number generation algorithm 108 to create the token 70 and associates the token 70 with the input value 60. Along these lines, the tokenization circuitry 50 securely stores the association between the token 70 and the input value 60 so that an attacker will be unable to discover the input value 60 if able to obtain the user token 70.

In step 306, the processing circuitry 42 collects user information (i.e., transaction details 104, also see FIG. 2) and associates the user information with the user token 70. Such collection occurs over a time range including periods of transaction inactivity and further transaction activity by the user 22 due to rotation of the user token 70 through different states of a token lifecycle (also see FIG. 3).

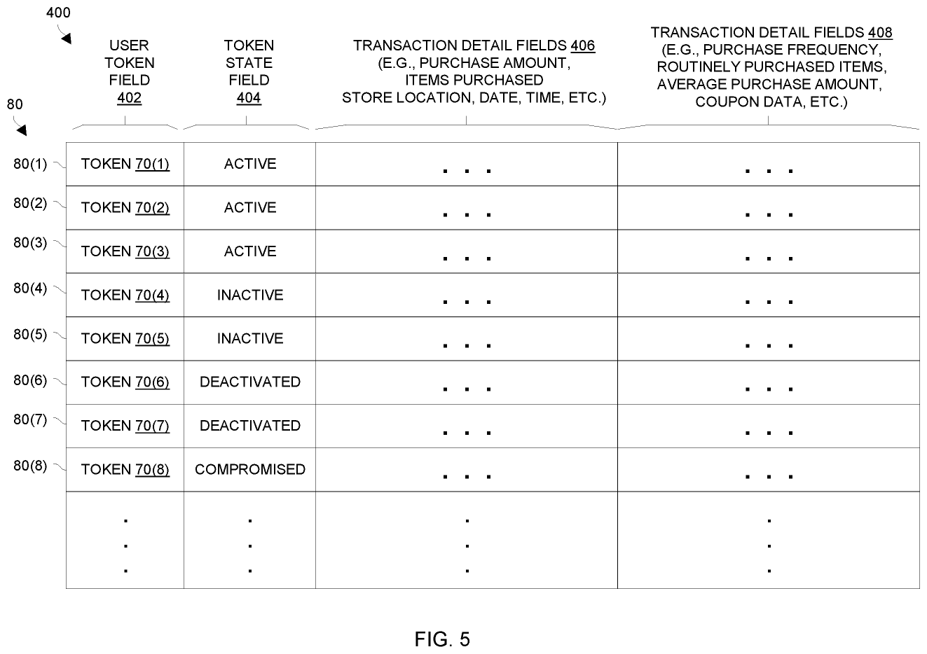

FIG. 5 shows a suitable structure 400 for information within the token database 44 (FIG. 1). As shown, each token database entry 80 includes a user token field 402, a token state field 404, a set of transaction detail fields 406, and a set of habit fields 408. It should be understood that the actual way the structure 400 is stored in computer memory may take a variety of forms (e.g., relational database, linked list, optimizations for binary searching, etc.).

For each entry 80, the user token field 402 stores a unique user token 70 in lieu of a user's input value 40 (i.e., potentially sensitive user information). As mentioned earlier, the tokens 70 are irreversible in the sense that an attacker cannot re-derive the input value 40 from the tokens 70.

The token state field 404 of each entry 80 stores the formal token state of the token 70 in the token field 402 of that entry 80. The particular token states may be represented by different digital values. Recall that FIG. 3 provided a state diagram which includes suitable states for the tokens 70. Other states are available for use as well (e.g., “dead”, “archived”, “special”, and so on).

The set of transaction detail fields 406 include application specific fields which may be of interest to the operator of the processing circuitry 42 (FIGS. 1 and 2). For example, in the context of a merchant, the fields 406 may include purchase amounts, a listing of purchased items, visited store locations, purchase dates/times, etc.

The set of habit fields 406 include processed information which the processing circuitry 42 has generated over time during periodic analysis. Again, in the context of a merchant, the fields 408 may include an aggregate purchase amount, a purchase frequency, a listing of commonly purchased items, information which could be used in promotions, etc.

It should be understood that modifications and enhancements can be made to the structure 400. For instance, a non-merchant application may not need certain fields 406, 408 and/or necessitate or desire other fields which are more appropriate for that application.

At this point, it should be further understood that the information contained within the structure 400 is relatively uninteresting to an attacker since it does not contain the user input values 40. As a result, such a structure 400 may be acceptable to maintain over an extended period of time while preserving compliance with data security rules and regulations (e.g., beyond legal time limits for security-sensitive data).

As described above, improved techniques are directed to managing user information (e.g., customer purchase habits) in an electronic system 20 which smartly applies a tokenization process over a time range which includes periods of user transaction activity and inactivity. In particular, the system 20 is equipped to associate initial user information (e.g., initial purchase information) with a token 70, and subsequently associate additional user information (e.g., subsequent purchase information) with the token 70 even if an extended amount of time has passed. Such operation is coordinated by rotating the token through various states of a token lifecycle. In some arrangements, the system implements formal mutually exclusive token states (e.g., “active”, “inactive”, “deactivated” and “compromised”) to smartly manage the user information over time.

While various embodiments of the invention have been particularly shown and described, it will be understood by those skilled in the art that various changes in form and details may be made therein without departing from the spirit and scope of the invention as defined by the appended claims.

For example, it should be understood that the various components described above can be located and under control of a variety of different entities. Along these lines the input devices 40 were described above as forming part of the same front-end subsystem 30 which has the processing circuitry 42 and the token database 44. In other arrangements, the input devices 40 may be separate from the processing circuitry 42 and the token database 44. For instance, in a healthcare context, the input devices 40 may be distribute among one or more of a variety of healthcare information sources (e.g., a doctor's office, a hospital, an employer, a payroll service, etc.) which generates healthcare transactions, and the back-end subsystem 32 may be a healthcare provider which processes the healthcare transactions. Since the processing circuitry 42 simply receives a user token 70 rather than healthcare transaction details of the user, the processing circuitry 42 is able to effectively administer render assistance to the user 22 (e.g., an employer or payroll service can provide a pay check with benefit information to an employee) without any of the healthcare transaction details.

It should be understood that other applications are suitable for use by the electronic system 20 as well. In particular, the electronic system 20 can be utilized in settings which involve security sensitive input values 60 from users 22, i.e., sequences of symbols such as credit card numbers, patient numbers, social security numbers, usernames, and so on.

What is claimed is:

1. A system comprising:
a front-end subsystem, located at a first location, constructed and arranged to provide data security at the first location by storing user transaction information into a token database contained in the front-end subsystem by storing the user transaction information in association with user tokens instead of user identification information; and
a back-end subsystem, located at a second location that is different from the first location, and communicably connected to the front-end subsystem by at least one computer network, constructed and arranged to generate the user tokens that are stored by the front-end subsystem into the token data base, to transmit the user tokens to the front-end subsystem over the computer network, and to store the tokens in association with the user identification information within a transaction processing database that is contained in the back-end subsystem.

2. The system of claim 1, wherein the user transaction information comprises security-sensitive non-public information;
wherein the user tokens generated by the back-end subsystem uniquely identify users and enable the front-end system to associate purchase habit data with the user tokens instead of the security-sensitive non-public information in the token database; and
wherein the front-end subsystem storing the user transaction information in association with the user tokens instead of the security-sensitive non-public information in the token database contained in the front-end subsystem maintains compliance with regulations regarding data security.

3. The system of claim 2, wherein the front-end subsystem is further constructed and arranged to receive the security-sensitive non-public information as input values from the users through an input device contained in the front-end system, and to transmit the input values to the back-end subsystem over the computer network; and
wherein the back-end subsystem is further constructed and arranged to generate each one of the user tokens in response to receipt of a corresponding one of the individual input values from the front-end subsystem over the computer network.

4. The system of claim 3, wherein the input device contained in the front-end subsystem comprise a credit card reader; and
wherein the security-sensitive non-public information received as input values from the users through the input device comprises credit card numbers.

5. The system of claim 3, wherein the back-end subsystem is further constructed and arranged to generate the user tokens using tokenization circuitry contained in the back-end subsystem; and
wherein the tokenization circuitry contained in the back-end subsystem is further constructed and arranged to use a random number generation algorithm to generate each user token. cm 6. The system of claim 5, wherein the tokenization circuitry contained in the back-end subsystem is further constructed and arranged to store each user token in association with a corresponding input value.

7. The system of claim 6, wherein the front-end subsystem is further constructed and arranged to transmit the input values to the back-end subsystem over the computer network in transaction messages containing the input values and transaction details identifying particular details of corresponding credit card transactions; and
wherein the back-end subsystem further contains processing circuitry constructed and arranged to process the credit card transactions by performing transactional operations based on the transactional details in the transaction messages received from the front-end subsystem and store processing results in a transaction processing database contained in the back-end subsystem.

8. The system of claim 7, wherein the front-end subsystem is further constructed and arranged to provide, for each token received by the front-end subsystem from the back-end subsystem over the computer network, a particular token state of the token from a group of mutually exclusive states, wherein the group of mutually exclusive states includes an active state, a plurality of non-active states, and a compromised state, wherein the active state and the non-active states are non-compromised states in the group of mutually exclusive states, and wherein the token is provided with the particular state from the group of mutually exclusive states at least in part by initially giving the token the “active” state from the group of mutually exclusive states, the “active” state indicating that a transaction associated with the input value has occurred within a predefined active state threshold amount of time.

9. The system of claim 8, wherein the front-end subsystem is further constructed and arranged to store the token state of each token in an entry for the token in the token database contained in the front-end subsystem, wherein the entry for the token stores both the token and the token state of the token.

10. The system of claim 9, wherein the front-end subsystem is further constructed and arranged to, for each token received by the front-end subsystem from the back-end subsystem over the computer network, in response to the token state for the token being currently equal to one of the non-compromised states in the group of mutually exclusive states, collect, in the entry for the token in the token database, user information, to associate the user information with the token during a time range which includes periods of transaction inactivity and further transaction activity by the user, wherein the user information comprises transaction details including date and time information for transactions performed using the same input value.

11. The system of claim 3, wherein the front-end subsystem is located at a first location comprising a merchant location;
wherein the back-end subsystem is located at a second location comprising a credit card transaction clearing center; and
wherein the at least one computer network communicably interconnects the merchant location and the credit card transaction clearing center.

12. The system of claim 5, wherein the tokenization circuitry contained in the back-end subsystem is further constructed and arranged to store each user token in association with the corresponding input value securely such that discovery of any input value based on the corresponding user token is prevented, and such that an attacker cannot discover any input value if able to obtain the corresponding user token.

13. The system of claim 12, wherein the tokenization circuitry contained in the back-end subsystem generates the user tokens without creating any cryptographic relationship between any input value and the corresponding user token.
